(19) United States
(12) Patent Application Publication
(10) Pub. No.: US 2020/0113700 A1
(43) Pub. Date: Apr. 16, 2020

(54) MEDICAL IMPLANTS WITH IMPROVED ROUGHNESS

(71) Applicant: THE REGENTS OF THE UNIVERSITY OF CALIFORNIA, Oakland, CA (US)

(72) Inventor: Takahiro OGAWA, Los Angeles, CA (US)

(21) Appl. No.: 16/626,273

(22) PCT Filed: Jun. 27, 2018
(86) PCT No.: PCT/US2018/039832

§ 371 Date: Dec. 23, 2019

Related U.S. Application Data
(60) Provisional application No. 62/526,202, filed on Jun. 28, 2017.

(51) Int. Cl. A61F 2/30; C23F 1/26; A61F 2/28

(52) U.S. Cl. CPC A61F 2/30771; C23F 1/26; A61F 2/28; A61F 2002/30838; A61F 2002/3084; A61F 2230/0026; A61F 2310/00407; A61L 2400/12; A61L 2400/18; A61F 2230/0086; A61F 2002/30925

(57) ABSTRACT

A medical implant has a hierarchical surface roughness and includes an implant body, which includes a combination of meso-scale surface features, micro-scale surface features, and nano-scale surface features.

### CROSS-REFERENCE TO RELATED APPLICATION

This application claims the benefit of U.S. Provisional Application No. 62/526,202, filed Jun. 28, 2017, the contents of which are incorporated herein by reference in their entirety.

### TECHNICAL FIELD

This disclosure generally relates to medical implants with improved roughness, including titanium and titanium alloy implants with such improved roughness.

### BACKGROUND

Degenerative change and injury of bone and joints and partially and fully edentulous jaw are on a rapid increase in an aging society. Implants formed of titanium or titanium alloys are used to repair, immobilize, stabilize, restore, and reconstruct these unhealthy, diseased, and defective areas. Titanium implants include screws, pins, plates, cages, and braces for spine and other areas of bone, artificial joints and stems for knee and hip areas, and dental implants in jaw and maxillofacial areas. Implant treatment faces many challenges of protracted healing time to integrate implants with bone, failure and revision surgery of implants, surgical complications, contraindications in patients with poor quality and quantity of bone and with adverse systemic conditions, post-surgery morbidity like inflammation and infection, insufficient mechanical tolerance and anchorage of implants, and so forth. Many, if not all, of these challenges and problems are closely associated with the insufficient capability of a titanium implant to allow growth of bone around the implant or adhere to the surrounding bone, namely the insufficient capability of bone-implant integration.

It is against this background that a need arose to develop the embodiments described herein.

### SUMMARY

In some embodiments, a medical implant has a hierarchical surface roughness and includes an implant body, which includes a combination of meso-scale surface features, micro-scale surface features, and nano-scale surface features.

In some embodiments of the medical implant, the meso-scale surface features have sizes in a range of about 5 μm to about 1 mm.

In some embodiments of the medical implant, the meso-scale surface features have sizes in a range of about 5 μm to about 200 μm.

In some embodiments of the medical implant, the meso-scale surface features include protruding structures. In some embodiments, the protruding structures have lateral sizes in a range of about 5 μm to about 200 μm, and heights in a range of about 5 μm to about 200 μm. In some embodiments, the protruding structures include cone-shaped structures, nodule-shaped structures, pyramid-shaped structures, stud- or spike-like trapezoidal structures, hemispherical structures, or hemispheroidal structures.

In some embodiments of the medical implant, the micro-scale surface features have sizes in a range of about 1 μm to about 5 μm.

In some embodiments of the medical implant, the nano-scale surface features have sizes in a range up to about 1 μm.

In some embodiments of the medical implant, the nano-scale surface features have sizes in a range of about 10 nm to about 1 μm.

In some embodiments of the medical implant, the nano-scale surface features include protruding structures. In some embodiments, the protruding structures include ridge-shaped, pillar/needle-like, or nodular structures.

In some embodiments of the medical implant, the nano-scale surface features include compartmental structures.

In some embodiments of the medical implant, the nano-scale surface features and the micro-scale surface features are superimposed onto the meso-scale surface features.

In some embodiments, the medical implant is a metallic implant. In some embodiments, the metallic implant is a titanium or titanium alloy implant.

In additional embodiments, a medical implant includes an implant body including a surface characterized by an average roughness (Rₐ) of about 1.5 μm or greater. In some embodiments, Rₐ is about 2 μm or greater. In some embodiments, Rₐ is about 2.5 μm or greater.

In additional embodiments, a medical implant includes an implant body including a surface characterized by an average peak-to-valley roughness (R_{z}) of about 7 μm or greater. In some embodiments, R_{z} is about 8 μm or greater. In some embodiments, R_{z} is about 10 μm or greater.

In additional embodiments, a medical implant includes an implant body including a surface characterized by an average slope of roughness profile (R_{δa}) of about 0.21 or greater. In some embodiments, R_{δa} is about 0.23 or greater. In some embodiments, R_{δa} is about 0.25 or greater.

In additional embodiments, a medical implant includes an implant body including a surface characterized by: (a) an average roughness (Rₐ) of about 1.5 μm or greater; (b) an average peak-to-valley roughness (R_{z}) of about 7 μm or greater; and (c) an average slope of roughness profile (R_{δa}) of about 0.21 or greater. In some embodiments, Rₐ is about 2.5 μm or greater, R_{z} is about 10 μm or greater, and R_{δa} is about 0.25 or greater.

In additional embodiments, a method of forming a medical implant having a hierarchical surface roughness includes subjecting the medical implant to surface treatment by exposing the medical implant to an etching liquid while generating bubbles within the etching liquid.

In some embodiments of the method, the etching liquid includes an acid.

In some embodiments of the method, exposing the medical implant to the etching liquid includes disposing an auxiliary material within the etching liquid, and disposing the medical implant over the auxiliary material. In some embodiments, the auxiliary material is or includes a same material as that of the medical implant. In some embodiments, the auxiliary material is or includes a different material as that of the medical implant. In some embodiments, the auxiliary material is or includes substantially pure titanium (e.g., purity of about 98% or greater, or about 99% or greater). In some embodiments, the auxiliary material is or includes substantially pure titanium, and the medical implant is a titanium or titanium alloy implant.

In some embodiments of the method, exposing the medical implant to the etching liquid is carried out at a temperature of about 140° C. or higher. In some embodiments, exposing the medical implant to the etching liquid also includes disposing an auxiliary material within the etching liquid, and disposing the medical implant over the auxiliary material.

In some embodiments of the method, exposing the medical implant to the etching liquid is carried out while agitating the etching liquid using an agitator. In some embodiments, the agitator is an ultrasonic device.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1 shows scanning electron microscopy (SEM) low-magnification images of micro-scale rough (A) and hierarchically rough (B) titanium surfaces.

FIG. 2 shows images of bird-eye views of micro-scale rough (A) and hierarchically rough (B) titanium surfaces.

FIG. 3 shows higher magnification SEM images of micro-scale rough (A) and hierarchically rough (B) titanium surfaces.

FIG. 4 shows even higher magnification SEM images of micro-scale rough (A) and hierarchically rough (B, C, and D) titanium surfaces.

FIG. 5 shows a large-scale (λ=250 μm) surface profiling of micro-scale rough (A) and hierarchically rough (B) titanium surfaces.

FIG. 6 shows a small-scale (λ=0.25 μm) surface profiling of micro-scale rough (A) and hierarchically rough (B) titanium surfaces.

FIG. 7 shows results of a quantitative measurement of surface roughness from a large-scale (λ=250 μm) surface profiling of micro-scale rough and hierarchically rough titanium surfaces.

FIG. 8 shows results of a quantitative measurement of surface roughness from a small-scale (λ=0.25 μm) surface profiling of micro-scale rough and hierarchically rough titanium surfaces.

FIG. 9 shows the strength of bone-implant integration evaluated by a biomechanical push-in test in a femoral bone.

FIG. 10 is a schematic of a medical implant having a hierarchical surface roughness.

FIG. 11 shows low-magnification SEM images (A, B), a mid-magnification SEM image (C), and a high-magnification SEM image (D) of a hierarchically rough titanium alloy surface.

### DETAILED DESCRIPTION

Besides various macroscopic designs, other titanium implants have micro-scale (scale between about 1 μm and about 5 μm) topography to promote bone-implant integration. To further improve titanium implants, adding meso-scale (scale between about 5 μm to about 200 μm) surface roughness can enhance a mechanical interlocking between titanium and bone, while adding nano-scale (scale up to about 1 μm or less) surface roughness can further promote the function of bone-forming cells. Here some embodiments are directed to the formation of an improved titanium surface with hierarchical morphology of meso-, micro- and nano-scale structures. Titanium implants with this hierarchical surface roughness have been demonstrated to show greater strength of bone-implant integration than titanium implants with micro-scale topography alone.

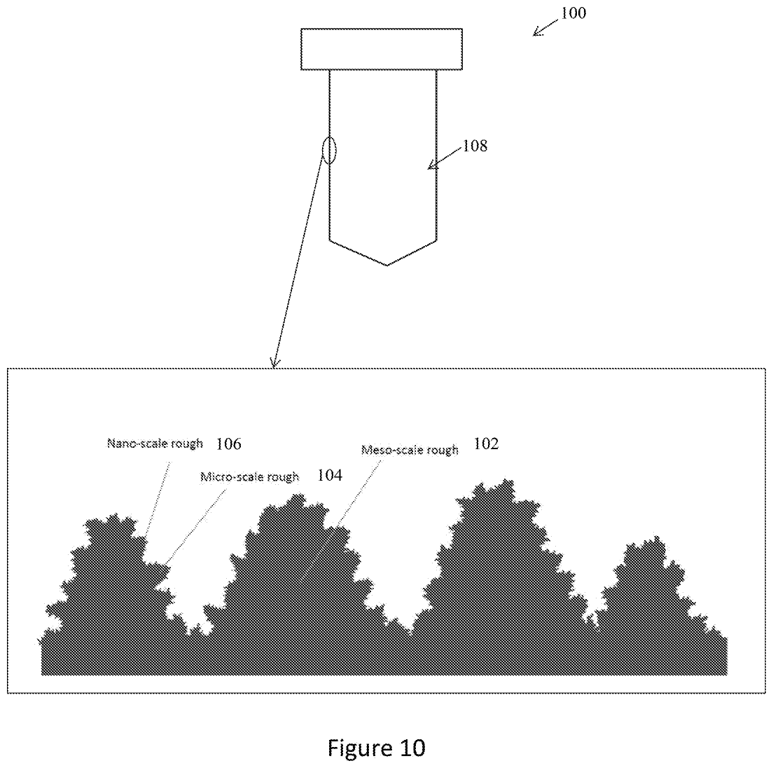

Provided herein are medical implants for enhancing bone-implant integration capabilities. FIG. 10 is a schematic of a medical implant 100 according to some embodiments. The medical implant 100 includes an implant body 108 which includes a surface topography having a hierarchical surface roughness deriving from the presence of a combination meso-scale surface features 102, micro-scale surface features 104, and nano-scale surface features 106. In some embodiments, the micro-scale surface features 104 include compartmental structures composed of projecting or protruding structures (e.g., in the form of peaks) at least partially surrounding respective areas (e.g., in the form of valleys) and having lateral sizes (or having an average lateral size), along a surface of the implant body 108, in a range of about 1 μm or greater, such as about 1 μm to about 5 μm Feret's diameter or longest diagonal dimension from a top view. In some embodiments, the meso-scale surface features 102 include projecting or protruding structures (e.g., in the form of cone-, nodule-, or pyramid-like or -shaped structures) having lateral sizes (or having an average lateral size), along the surface of the implant body 108, greater than that of the micro-scale surface features 104 and in a range of about 1 μm or greater, such as about 5 μm to about 1 mm, about 5 μm to about 800 μm, about 5 μm to about 600 μm, about 5 μm to about 400 μm, about 5 μm to about 200 μm, or about 20 μm to about 100 μm in Feret's diameter, and having heights (or having an average height), extending from the surface of the implant body 108, in a range of about 1 μm or greater, such as about 5 μm to about 1 mm, about 5 μm to about 800 μm, about 5 μm to about 600 μm, about 5 μm to about 400 μm, about 5 μm to about 200 μm, about 20 μm to about 100 μm, or about 20 μm to about 40 μm, and having aspect ratios (or having an average aspect ratio), given as a ratio of a height extending from the surface to a lateral size along the surface, in a range of about 0.1 to about 5, about 0.1 to about 3, about 0.1 to about 2, about 0.2 to about 2, or about 0.5 to about 1.5. In some embodiments, the nano-scale surface features 106 include projecting or protruding structures (e.g., in the form of ridge-shaped, pillar/needle-like, and nodular structures) having lateral sizes (or having an average lateral size), along the surface of the implant body 108, smaller than that of the micro-scale surface features 104 and in a range up to about 1 μm, such as about 1 nm to about 1 μm, about 10 nm to about 1 μm, about 10 nm to about 100 nm, about 100 nm to about 1 μm, about 100 nm to about 500 nm, or about 500 nm to about 1 μm. In some embodiments, the nano-scale surface features 106 also include compartmental structures composed of projecting or protruding structures (e.g., in the form of peaks) at least partially surrounding respective areas (e.g., in the form of valleys) and having sizes (or having an average size), along the surface of the implant body 108, in a range of up to about 1 μm, such as about 1 nm to about 1 μm, about 10 nm to about 1 μm, about 10 nm to about 100 nm, about 100 nm to about 1 μm, about 100 nm to about 500 nm, or about 500 nm to about 1 μm. In some embodiments, the nano-scale surface features 106 and the micro-scale surface features 104 are superimposed onto, incorporated onto, or disposed on the meso-scale surface features 102.

In some embodiments, the medical implant 100 having the hierarchical surface roughness is characterized by, according to surface profiling using a profilometer, one or a combination of two or more of the following: (1) an average roughness (Rₐ) (arithmetic average of absolute values of vertical deviations of a surface profile about a mean line within a sampling length) in a range of about 1.2 μm or greater, such as about 1.3 μm or greater, about 1.5 μm or greater, about 1.8 μm or greater, about 2 μm or greater, about 2.3 μm or greater, or about 2.5 μm or greater, and up to about 8 μm or greater, or up to about 10 μm or greater; (2) an average peak-to-valley roughness (R_{z} or Rₚ₋ᵥ) (arithmetic average of vertical distances between peaks and valleys of a surface profile within a sampling length) in a range of about 6.5 μm or greater, such as about 7 μm or greater, about 7.5 μm or greater, about 8 μm or greater, about 8.5 μm or greater, about 9 μm or greater, about 9.5 μm or greater, or about 10 μm or greater, and up to about 15 μm or greater, or up to about 20 μm or greater; and (3) an average slope of roughness profile (R_{δa}) (arithmetic average of absolute values of a slope of a surface profile within a sampling length) in a range of about 0.2 or greater, such as about 0.21 or greater, about 0.22 or greater, about 0.23 or greater, about 0.24 or greater, or about 0.25 or greater, and up to about 0.5 or greater, or up to about 0.8 or greater.

In some embodiments, the medical implant 100 is a metallic implant including one or more metals, such as a titanium implant. Other examples of metallic implants include titanium alloy implants, chromium-cobalt alloy implants, platinum and platinum alloy implants, nickel and nickel alloy implants, stainless steel implants, zirconium implants, zirconia implants, titanium-zirconia alloy implants, gold or gold alloy implants, and aluminum or aluminum alloy implants. In other embodiments, the medical implant 100 is a non-metallic implant. Examples of non-metallic implants include ceramic implants, calcium phosphate implants, and polymeric implants.

Also provided herein are methods of forming medical implants having a hierarchical surface roughness. In some embodiments, a method includes subjecting a medical implant to surface treatment by exposing the medical implant to an etching liquid while generating bubbles within the etching liquid. In some embodiments, the etching liquid includes an acid, such as sulfuric acid (H₂SO₄) or another strong acid. In some embodiments, exposing the medical implant to the etching liquid is carried out for a time period in a range of 5 seconds to about 10 minutes, such as about 5 seconds to about 5 minutes, about 10 seconds to about 5 minutes, about 10 seconds to about 3 minutes, or about 10 seconds to about 2 minutes. In some embodiments, exposing the medical implant to the etching liquid is carried out at a temperature in a range of about 100° C. to about 140° C., such as about 110° C. to about 130° C., or about 120° C. Other manners of surface treatment are contemplated in place of, or in combination with, acid treatment, such as alkaline treatment, oxidation, light irradiation, material deposition (e.g., sputtering, plasma spraying, or vapor deposition), and physical treatments like laser-etching, machining, or sandblasting. For example, laser-etching can be performed to yield meso-scale surface features, along with acid treatment to yield micro- and nano-scale surface features.

In some embodiments, generation of bubbles is promoted by disposing an auxiliary material within the etching liquid, such as within a container, and disposing the medical implant over the auxiliary material. In some embodiments, the auxiliary material is or includes a same or similar material as that of the medical implant, such as titanium or another metal or combination of metals. In some embodiments, the auxiliary material is or includes a different material as that of the medical implant. The auxiliary material can be in the form of fibers, a rod, a wire, an array, coil, or stack of the foregoing, or a disk or a cylinder. In some embodiments, the inclusion of the auxiliary material promotes vigorous, accelerated, and enhanced flow of the etching liquid and promotes generation of bubbles. In some embodiments, the bubbles are generated via a chemical reaction between the auxiliary material and the etching liquid, and at least some of the bubbles impinge upon or bombard a surface of the medical implant to impart a hierarchical surface roughness.

In some embodiments, generation of bubbles is promoted by exposing the medical implant to the etching liquid at an elevated temperature in a range of about 140° C. or higher, such as about 140° C. to about 160° C., or about 140° C. In some embodiments, the elevated temperature promotes vigorous, accelerated, and enhanced flow of the etching liquid and promotes generation of bubbles. In some embodiments, the bubbles are generated via a chemical reaction between the medical implant and the etching liquid, and at least some of the bubbles impinge upon or bombard the surface of the medical implant to impart the hierarchical surface roughness. In some embodiments, generation of the bubbles is promoted by exposing the medical implant to the etching liquid at an elevated temperature, along with disposing an auxiliary material within the etching liquid.

Other manners of promoting the flow of the etching liquid and generation of bubbles are contemplated, such as by agitating the etching liquid using a mechanical agitator, such as an ultrasonic device or a mechanical vibrator. Also, combinations of two or more of the foregoing surface treatments are contemplated, such as using two or more of an auxiliary material, an elevated temperature, material deposition, laser-etching, and agitation.

In some embodiments, the medical implant (or another reference medical implant) prior to surface treatment has, according to a large scale (λ=250 μm) surface profiling, a reference average roughness (Rₐ), a reference maximum peak-to-valley roughness (Rₘₐₓ), a reference average width of roughness profile elements (Rₛₘ), a reference skewness of roughness profile (Rₛₖ), and a reference kurtosis of roughness profile (Rₖᵤ), and the medical implant subsequent to surface treatment has, according to the large scale (λ=250 μm) surface profiling, an average roughness (Rₐ) greater than the corresponding reference value, a maximum peak-to-valley roughness (Rₘₐₓ) greater than the corresponding reference value, an average width of roughness profile elements (Rₛₘ) greater than the corresponding reference value, a skewness of roughness profile (Rₛₖ) smaller than the corresponding reference value, and a kurtosis of roughness profile (Rₖᵤ) smaller than the corresponding reference value.

In some embodiments, the medical implant (or another reference medical implant) prior to surface treatment has, according to a small scale (λ=0.25 μm) surface profiling, a reference average roughness (Rₐ), a reference maximum peak-to-valley roughness (Rₘₐₓ), a reference average width of roughness profile elements (Rₛₘ), a reference skewness of roughness profile (Rₛₖ), and a reference kurtosis of roughness profile (Rₕ), and the medical implant subsequent to surface treatment has, according to the small scale (λ=0.25 μm) surface profiling, an average roughness (Rₐ) smaller than the corresponding reference value, a maximum peak-to-valley roughness (Rₘₐₓ) smaller than the corresponding reference value, an average width of roughness profile elements (Rₛₘ) smaller than the corresponding reference value, a skewness of roughness profile (Rₛₖ) smaller than the corresponding reference value, and a kurtosis of roughness profile (Rₖᵤ) smaller than the corresponding reference value.

The medical implant provided herein can be used for treating, preventing, ameliorating, correcting, or reducing one or more symptoms of a medical condition by implanting the medical implant in a mammalian subject. The mammalian subject can be a human or a veterinary animal such as a dog, a cat, a horse, a cow, a bull, or a monkey. Examples of medical conditions that can be treated or prevented include missing teeth or bone related medical conditions such as femoral neck fracture, orthodontic anchorage, wrist fracture, spine fracture/disorder, spinal disk displacement, edentulous jaw, fracture or degenerative changes of joints such as knee joint arthritis, bone and other tissue defect or recession caused by a disorder or a body condition such as cancer, injury, systemic metabolism, infection or aging, and combinations thereof.

### EXAMPLES

The following examples describe specific aspects of some embodiments of this disclosure to illustrate and provide a description for those of ordinary skill in the art. The examples should not be construed as limiting this disclosure, as the examples merely provide specific methodology useful in understanding and practicing some embodiments of this disclosure.

### Example 1

Results

Surface Morphology of Micro-Scale Rough Titanium and Hierarchically Rough Titanium Surfaces

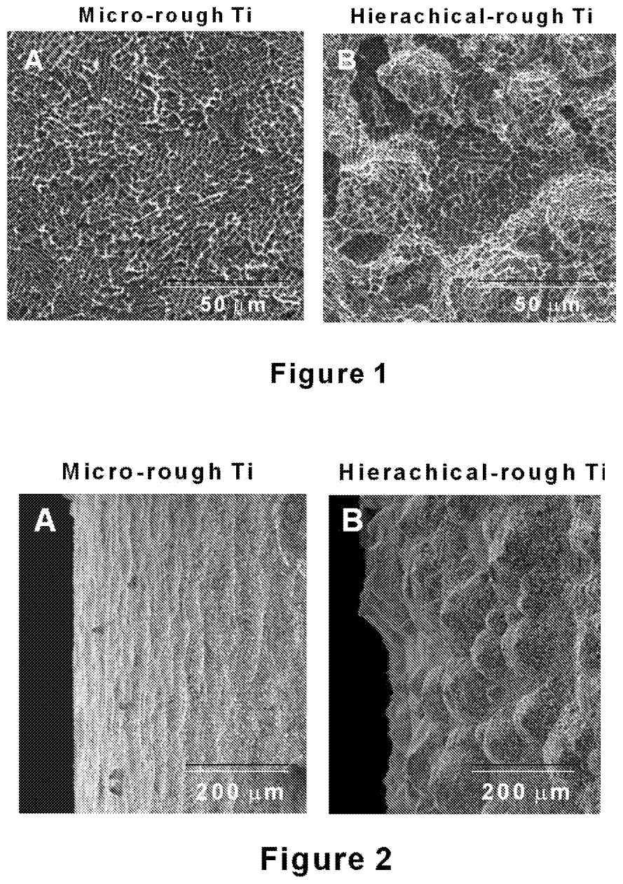

FIG. 1 shows scanning electron microscopy (SEM) low-magnification images of micro-scale rough and hierarchically rough titanium surfaces. The micro-scale rough titanium shows representative surface features typically observed on comparative titanium implants, composed of uniformly created compartmental structures at the micron level of a few microns without a larger scale structure (FIG. 1A). The surface is seen as relatively flat even at this magnification. The hierarchically rough titanium shows meso-scale (about 5 μm to about 200 μm in Feret's diameter or longest diagonal dimension) cone-, nodule-, pyramid-like, trapezoidal, hemispherical, or hemispheroidal projecting or protruding structures simultaneously with uniformly created micro-scale compartmental structures similarly seen on the micro-scale rough titanium (FIG. 1B).

FIG. 2 shows images of bird-eye views of the two different titanium surfaces, confirming the presence of micro-scale structures alone on the micro-scale rough titanium (FIG. 2A) and co-existence of micro-scale structures and meso-scale projecting structures on the hierarchically rough titanium surface (FIG. 2B).

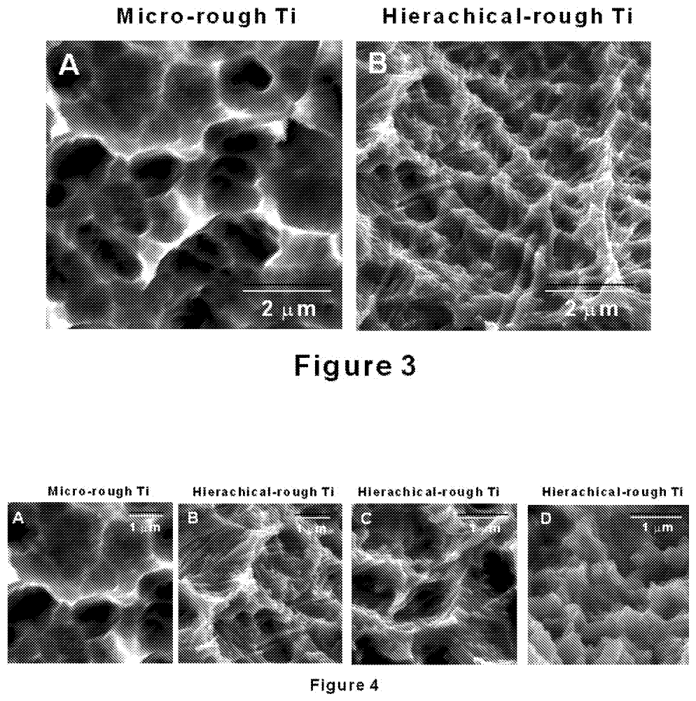

FIG. 3 shows higher magnification SEM images of the two titanium surfaces. The micro-scale rough titanium has a micro-scale compartmental topography composed of sharp peaks and valleys (FIG. 3A). The size of the compartments was between about 1 μm to about 5 μm in Feret's diameter. There are no definable structures smaller than the micro-scale compartments. The hierarchically rough titanium shows micro-sale and smaller scale compartments, ranging from about 100 nm to about 5 μm (FIG. 3B). The peaks are less sharp than those on the micro-scale rough titanium.

FIG. 4 shows even higher magnification SEM images. The micro-scale rough titanium was confirmed to have no definable nano-scale structures within the micro-scale compartments (FIG. 4A). The hierarchically rough titanium showed random polymorphic nano-scale structures within the compartments and along the peaks. The nano-scale structures included ridge-shaped structures, pillar/needle-like structures, and nodular structures with sizes in the range of about 10 nm to about 1000 nm (FIG. 4B-D).

Surface Profiles of Micro-Scale Rough Titanium and Hierarchically Rough Titanium Surfaces

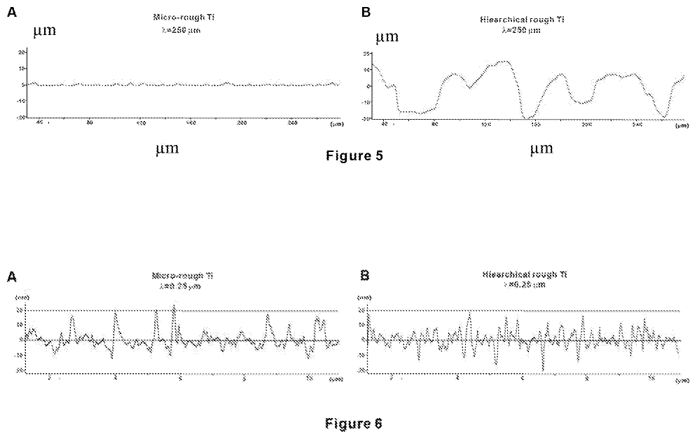

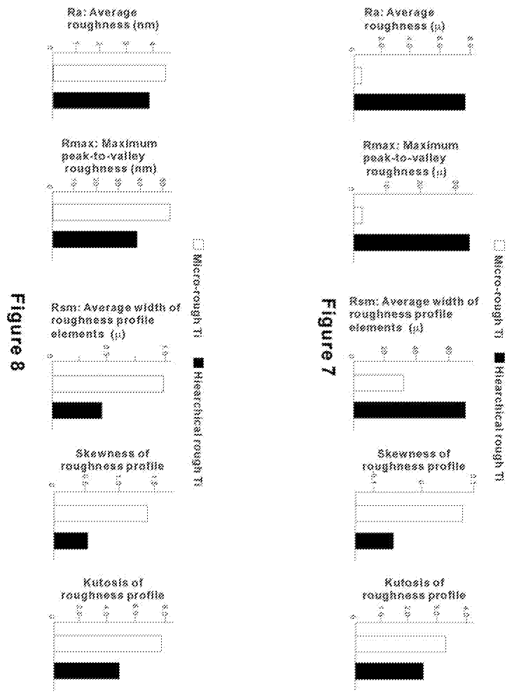

FIG. 5 shows a large-scale (λ=250 μm) surface profiling of the two titanium surfaces. The cross-sectional morphology of the micro-scale rough titanium was near flat at this scale, without showing a meso-scale structure (FIG. 5A). The hierarchically rough titanium showed fluctuating outlines, depicting meso-scale structures in the range of about 5 μm to about 200 μm in Feret's diameter and in the range of about 5 μm to about 200 μm in height (FIG. 5B).

FIG. 6 shows a small-scale (λ=0.25 μm) surface profiling of the two titanium surfaces. The micro-scale rough titanium showed peaks and valleys that form the compartmental structures (FIG. 6A). The inter-peak distance was generally about 1 μm or greater. The hierarchically rough titanium showed a denser oscillation of peaks and valleys, with its inter-peak distance being smaller than about 1 μm (FIG. 6B). This corroborated the above-shown submicron-sized compartments and nano-scale structures in SEM images of the hierarchically rough titanium.

Roughness and Profile Parameters of Micro-Scale Rough Titanium and Hierarchically Rough Titanium Surfaces

FIG. 7 shows results of a quantitative measurement of surface roughness of the two titanium surfaces. In the result from a large-scale (λ=250 μm) surface profiling, the hierarchically rough titanium showed a remarkably greater average roughness (Rₐ) and maximum peak-to-valley roughness (Rₘₐₓ), confirming the considerably enhanced meso-scale roughness than the micro-scale rough titanium. The hierarchically rough titanium also showed a substantially greater average width of roughness profile elements (Rₛₘ). The presence of meso-scale structures has contributed to this great inter-structure distance. The skewness of roughness profile (Rₛₖ) for the hierarchically rough titanium is lower than that for micro-scale rough titanium and lower than 0, indicating that the surface topography is skewed upwardly relative an average line. This indicates that the volume of projecting structures is larger than that of recessing structures. The kurtosis of roughness profile (Rₖᵤ), which is lower for the hierarchically rough titanium and even lower than about 3.0, indicated that the structures on the hierarchically rough titanium were less sharp for their height distribution than those on the micro-scale rough titanium.

FIG. 8 shows a continued surface roughness assessment of the two titanium surfaces in a small-scale (λ=0.25 μm). It was noted that Rₛₘ showed a great contrast between the large- and small-scale analyses. In this small-scale analysis, Rₛₘ was smaller for the hierarchically rough titanium than the micro-scale rough titanium. Rₛₘ for the micro-scale rough titanium was about 1 μm, whereas the one for the hierarchically rough titanium was smaller than about 0.5 μm—about half the value of the micro-scale rough titanium. This result confirmed the SEM observation that the compartmental structures are in a smaller scale on the hierarchically rough titanium and that there are nano-scale structures on the hierarchically rough surface. Rₖᵤ was smaller for the hierarchically rough surface, indicating that the structures on its surface are more rounded.

Strength of Bone-Implant Integration for Micro-Scale Rough Titanium and Hierarchically Rough Titanium

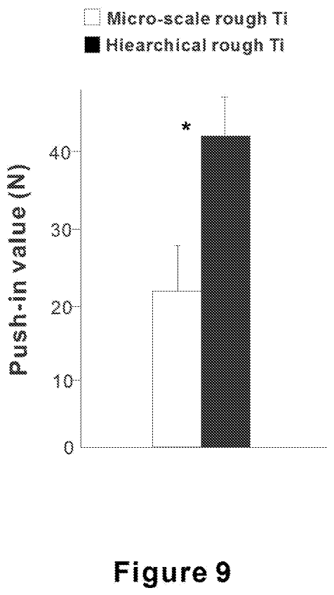

FIG. 9 shows the strength of bone-implant integration evaluated by a biomechanical push-in test in a femoral bone. The strength of bone-implant integration was about 2 times greater for the hierarchically rough titanium than for the micro-scale rough titanium.

Methods to Form Hierarchically Rough Titanium Surface

Method 1: Using Auxiliary Titanium

About 30 ml of H₂SO₄ (about 66% concentration) is heated to about 120° C. Commercially pure titanium wire (about 1 mm diameter, about 90 cm in length) in a coil form is submerged in the heated H₂SO₄. After confirming a chemical reaction is started and bubbles are vigorously generated, titanium implants or other titanium samples of interest are soaked into H₂SO₄ for about 75 seconds. Titanium implants are removed and rinsed with double-distilled H₂O.

Method 2: Using High-Temperature Acid

About 30 ml of H₂SO₄ (about 66% concentration) was heated to about 140° C. or higher. Titanium implants or other titanium samples of interest are soaked into H₂SO₄ for about 75 seconds. Titanium implants are removed and rinsed with double-distilled H₂O.

### Example 2

Surface profiling was performed for a hierarchically rough titanium surface and comparative titanium surfaces (under a condition with a sampling length of interest being 4 mm and a threshold of 0.8).

The hierarchically rough titanium showed a remarkably greater average roughness (Rₐ) of 2.8468±0.090 μm, in comparison with Rₐ of 0.3814±0.0233 μm for a comparative acid-etched surface, Rₐ of 0.8508±0.060 μm for a comparative sand-blasted surface, and Rₐ of 1.056±0.094 μm for a comparative sand-blasted and acid-etched surface.

The hierarchically rough titanium showed a remarkably greater average peak-to-valley roughness (R_{z} or Rₚ₋ᵥ) of 13.985±0.259 μm, in comparison with R_{z} of 1.933±0.147 μm for the comparative acid-etched surface, R_{z} of 4.790±0.392 μm for the comparative sand-blasted surface, and R_{z} of 5.931±0.543 μm for the comparative sand-blasted and acid-etched surface.

The hierarchically rough titanium showed a remarkably greater average slope of roughness profile (R_{δa}) of 0.3692±0.0088, in comparison with R_{δa} of 0.0656±0.0035 for the comparative acid-etched surface, R_{δa} of 0.1504±0.0097 for the comparative sand-blasted surface, and R_{δa} of 0.1854±0.0139 for the comparative sand-blasted and acid-etched surface.

### Example 3

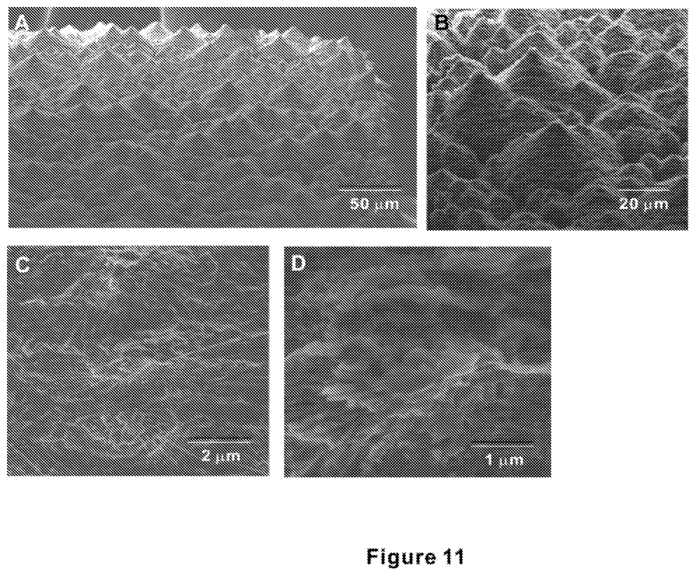

A hierarchically rough surface was formed on titanium alloy (Ti-6Al-4V). Specifically, a titanium alloy rod was treated with acid using titanium as an auxiliary material. FIG. 11A, B shows low-magnification SEM images after the treatment, depicting the formation of meso-scale spike-like, cone-shaped, or pyramid-shaped structures. FIG. 11C shows a mid-magnification SEM image, depicting the formation of micro-scale surface compartmental structures composed of peaks and valleys. FIG. 11D shows a high-magnification SEM image, depicting the formation of nano-scale surface morphology composed of nano-sized ridges, pillars, and nodules.

As used herein, the singular terms “a,” “an,” and “the” may include plural referents unless the context clearly dictates otherwise. Thus, for example, reference to an object may include multiple objects unless the context clearly dictates otherwise.

As used herein, the term “set” refers to a collection of one or more objects. Thus, for example, a set of objects can include a single object or multiple objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, concentrations, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a range of about 1 to about 200 should be understood to include the explicitly recited limits of about 1 and about 200, but also to include individual values such as about 2, about 3, and about 4, and sub-ranges such as about 10 to about 50, about 20 to about 100, and so forth.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations are not a limitation of the disclosure.

What is claimed is:

1. A medical implant having a hierarchical surface roughness, comprising:
an implant body including a combination of meso-scale surface features, micro-scale surface features, and nano-scale surface features.

2. The medical implant of claim 1, wherein the meso-scale surface features have sizes in a range of 5 μm to 1 mm.

3. The medical implant of claim 2, wherein the meso-scale surface features have sizes in a range of 5 μm to 200 μm.

4. The medical implant of claim 1, wherein the meso-scale surface features include protruding structures.

5. The medical implant of claim 4, wherein the protruding structures have lateral sizes in a range of 5 μm to 200 μm, and heights in a range of 5 μm to 200 μm.

6. The medical implant of claim 4, wherein the protruding structures include cone-shaped, nodule-shaped, pyramid-shaped, trapezoidal, hemispherical, or hemispheroidal structures.

7. The medical implant of claim 1, wherein the micro-scale surface features have sizes in a range of 1 μm to 5 μm.

8. The medical implant of claim 1, wherein the nano-scale surface features have sizes in a range up to 1 μm.

9. The medical implant of claim 8, wherein the nano-scale surface features have sizes in a range of 10 nm to 1 μm.

10. The medical implant of claim 1, wherein the nano-scale surface features include protruding structures.

11. The medical implant of claim 1, wherein the nano-scale surface features include compartmental structures.

12. The medical implant of claim 1, wherein the nano-scale surface features and the micro-scale surface features are superimposed onto the meso-scale surface features.

13. The medical implant of claim 1, wherein the medical implant is a metallic implant.

14. The medical implant of claim 13, wherein the metallic implant is a titanium or titanium alloy implant.

15. A medical implant, comprising:
an implant body including a surface characterized by an average roughness (Rₐ) of 1.5 μm or greater.

16. The medical implant of claim 15, wherein Rₐ is 2 μm or greater.

17. The medical implant of claim 15, wherein Rₐ is 2.5 μm or greater.

18. A medical implant, comprising:
an implant body including a surface characterized by an average peak-to-valley roughness (R_{z}) of 7 μm or greater.

19. The medical implant of claim 18, wherein R_{z} is 8 μm or greater.

20. The medical implant of claim 18, wherein R_{z} is 10 μm or greater.

21. A medical implant, comprising:
an implant body including a surface characterized by an average slope of roughness profile (R_{δa}) of 0.21 or greater.

22. The medical implant of claim 21, wherein R_{δa} is 0.23 or greater.

23. The medical implant of claim 21, wherein R_{δa} is 0.25 or greater.

24. A medical implant, comprising:
an implant body including a surface characterized by:
(a) an average roughness (Rₐ) of 1.5 μm or greater;
(b) an average peak-to-valley roughness (R_{z}) of 7 μm or greater; and
(c) an average slope of roughness profile (R_{δa}) of 0.21 or greater.

25. The medical implant of claim 24, wherein Rₐ is 2.5 μm or greater, R_{z} is 10 μm or greater, and R_{δa} is 0.25 or greater.

26. A method of forming a medical implant having a hierarchical surface roughness, comprising subjecting the medical implant to surface treatment by exposing the medical implant to an etching liquid while generating bubbles within the etching liquid.

27. The method of claim 26, wherein the etching liquid includes an acid.

28. The method of claim 26, wherein exposing the medical implant to the etching liquid includes disposing an auxiliary material within the etching liquid, and disposing the medical implant over the auxiliary material.

29. The method of claim 28, wherein the auxiliary material is the same as a material of the medical implant.

30. The method of claim 28, wherein the auxiliary material is different than a material of the medical implant.

31. The method of claim 26, wherein exposing the medical implant to the etching liquid is carried out at a temperature of 140° C. or higher.

32. The method of claim 31, wherein exposing the medical implant to the etching liquid further includes disposing an auxiliary material within the etching liquid, and disposing the medical implant over the auxiliary material.

33. The method of claim 26, wherein exposing the medical implant to the etching liquid is carried out while agitating the etching liquid using an agitator.

34. The method of claim 33, wherein the agitator is an ultrasonic device.
